(19) United States
(12) Patent Application Publication
(10) Pub. No.: US 2012/0107253 A1
(43) Pub. Date: May 3, 2012

(54) SUNSCREEN FORMULATIONS USING NATURAL OCEANIC CLAY (aka Marine Glacial Clay)

(76) Inventors: HOLLY XING, RICHMOND (CA); TIMOTHY GARLAND, DELTA (CA)

(21) Appl. No.: 13/279,305

(22) Filed: Oct. 23, 2011

Related U.S. Application Data
(60) Provisional application No. 61/409,432, filed on Nov. 2, 2010.

(51) Int. Cl. A61K 8/27; A61Q 17/04; A61K 8/19

(52) U.S. Cl. 424/59

(57) ABSTRACT

Sunscreen formulations use the Natural Oceanic Clay when combined with sunscreen actives such as zinc oxide, can provide significant enhancement in absorbance of UVA, UVB, and UVC, with increased SPF and PFA readings.

### CROSS REFERENCE TO OTHER APPLICATIONS

This application claims the benefit of U.S. Provisional Patent Application No. 61/409,432 filed in the USPTO on Nov. 2, 2010 and having the same title and inventors as the present invention.

### FEDERAL FUNDING

N/A

### FIELD OF THE INVENTION

This invention relates to topical sun or radiation screening and more particularly to a sun protection formulation using Natural Oceanic Clay (aka Marine Glacial Clay) as a highly effective SPF (Sun Protection Factor for UVB) & PFA (Protection factor UVA) boosting agent, as well as providing protection against UVC

### BACKGROUND OF THE INVENTION

Skin cancer is the most common cancer in the world today. The two most common variants of skin cancer are basal and squamous cell carcinoma. The most important risk factor for skin cancer is exposure to the sun. Sunscreen formulations are well known in the art. Typically, the commercial variety of modern sunscreen formulations are adapted to protect against broad spectrum UV radiation by using physical and chemical blockers. Physical blockers such as zinc oxide and titanium oxide reflect UV radiation away from the skin. Chemical blockers, both natural and synthetic act to absorb UV radiation. Modern sunscreen formulations have a number of disadvantages which can be itemized as follows:

1. They may contain zinc oxide or titanium oxide nanoparticles to permit transparency of the formulation on the skin. There are concerns that these nano-sized compounds can induce free radical formation in the presence of light and that this may damage these cells (photo-mutagenicity with zinc oxide). It's very difficult to obtain high SPF (45+) form using only Zinc Oxide or Titanium Dioxide, and the cost of these specialized sunscreen actives can be prohibitive as well.
2. A high amount of oil and chemical carriers are often used to dissolve and disperse sunscreen actives and improve the texture of the sunscreen. Sunscreen carrier systems can be highly irritating and comedogenic or even toxic due to their ability to generate free radicals and accumulate in human fatty tissues. Some carriers have estrogenic and carcinogenic properties.
3. Many chemical sunscreen actives and dispersing agents may be used and are potentially toxic to the human body. Such chemicals may cause photosensitization and make the skin even more sensitive to sunlight.
4. Many sunscreen actives such as Titanium Dioxide can be photo-unstable and so are often coated with alumina/stearic acid to promote stability when exposed to sunlight. These chemicals are also potentially toxic to the human body.

A significant amount of research has been done on the toxicity of sunscreens to the human body and to other animal life forms. Given the on-going problems associated with modern commercial sunscreens there is a need for a sunscreen that can be applied without fear of toxicity and is effective across a broad range of the UV spectrum.

### OBJECTS AND ADVANTAGES OF THE INVENTION

One object of the invention is to provide a sunscreen that is, highly effective

Another object of the present invention is to provide a sunscreen that complies with natural and organic cosmetic standards such as Ecocert and NSF International Standard/American National Standard so that it is organic, “green” and biodegradable.

Another object of the invention is to provide a sunscreen that uses Ecocert-approved natural preservative systems.

Yet another object of the invention is to provide a sunscreen that provides a high SPF in the range of SPF 45-55 and high PFA 24-32

Another object of the invention is to provide sun protection formulations that provide protection across a broad spectrum of UVA, UVB and UVC radiation.

One advantage of the present invention is that it does not use nanoparticles.

Another advantage of the present invention is that it is useful on the sensitive skin of children and does not irritate the skin or sting the eyes.

Another advantage of the invention is to provide sunscreens with esthetically desirable soft, smooth creamy texture and light, moisturizing, transparent, matt finish skin feel without oiliness

A further advantage of the present invention is that it uses the Natural Oceanic Clay in combination with a non-nanoparticle based zinc oxide.

One advantage of using Natural Oceanic Clay is that it significantly reduces the cost of the sunscreen formulation.

Yet another advantage of the present invention is that it can be formulated with pharmaceutical actives specifically for skin conditions such as acne, dermatitis, anti-fungal, anti-inflammatory.

### DESCRIPTION OF THE INVENTION

The following description of the present invention is merely exemplary in nature and not intended to limit the invention or the application or uses of the invention.

The present invention relates to a novel and inventive sunscreen compound that provides a high degree of protection against a broad spectrum of UV radiation and overcomes the deficiencies of prior art sunscreen formulations noted above.

Accordingly the present invention provides a sunscreen formulation for application to the skin comprising the Natural Oceanic Clay combined with a sunscreen active

### Natural Oceanic Clay (Marine Glacial Clay)

Clay has been used around the world for centuries by indigenous people as an important medicinal and cosmetic tool. Recently, medical research has emerged which supports the efficacy of clay and it is now becoming increasingly popular as a rediscovered treatment for many different health and skincare conditions. Externally, clays are used to absorb excess oil, dirt, and toxins from the skin while simultaneously exfoliating and improving skin circulation. Some clay, such as bentonite clay, is primarily ingested for medicinal purposes such as detoxification or mineral deficiencies. Other clays, such as French Green clay and Rhassoul clay, are used externally for skin conditions and for cosmetic purposes. Clays come in a variety of colors such as red, green, white, gray, and can range in texture from coarse and heavy to fine and fluffy. The different colors of clays occur because of their natural mineral content.

Cosmetic clays are made up of different mineral contents, and each clay type has a different effect on the skin. The high mineral content of clay rejuvenates the skin while the clay exfoliates and stimulates blood circulation to the skin. Regular use of clay facials will remove dead skin cells, improve circulation to the skin, remove debris from the pores and bring about a smooth healthy glow. Each clay has the ability to absorb toxins from the skin but they differ in their level of absorption. Clays contain massive amounts of trace minerals, necessary for good health. This may explain many of the healing properties of clay. Specific trace minerals that various clays possess vary very widely. Also, the amount of any particular trace mineral in any specific clay varies a lot among clays from different locations. For example, the amount of iron in various bentonite clays can vary from well below 1%, and up to 10%.

The clay of the present invention is a cosmetically acceptable Natural Oceanic Clay defined as chlorite-mica. The clay has a high iron, silica, calcium and magnesium content as well as many other nourishing minerals. Within the clay iron oxide, titanium dioxide and zinc oxide are found. The silica act to provide a physical barrier to the sun. The silica assists in the dispersion of the iron oxide particles within the clay when applied to human skin. The clay of the present invention has the following physiochemical properties:
Rich in Minerals: This all-natural oceanic clay has been maturing off the West Coast of Canada over thousands of years since formed by the movement of glaciers. It contains more than 70 minerals and rare earths, which is unique to this region of the world.
Balanced Ion Exchange Capacity: Our Natural Oceanic Clay is a powerful ion exchanger. When applied to the skin, the impurities, which are positively charged, get bound to the clay's powerful, negative charge, acting much like a magnet. The clay both cleans and detoxifies your skin.
High Astringency: After removing the impurities such as oil and dirt from the pores, Oceanic Clay also helps to reduce pore size, leaving the skin porcelain smooth.
Osmotic Power: The Oceanic Clay also uses its osmotic strength to pull moisture up from deep inner tissues to hydrate the face. This Natural Oceanic Clay cleans the skin but does not dehydrate it as other cleansing agents may do.
Colloidal Particles: Due to their minute sizes, Oceanic Clay's particles act as an emulsifying agent by forming a barrier around the oily particles,
Large surface area with high sorption capability: with surface area of 6000 square feet per gram, the clay acts as dermatological protector, with anti-inflammatory properties, cooling and soothing the skin with each application.
Balanced pH: Natural Oceanic Clay maintains a balanced pH on the skin with powerful buffering capability. Helps to strengthen the barrier function of the skin to keep it healthy and beautiful.

The Natural Oceanic Clay of the present invention has been found provide the following advantages:
Unprecedented broad spectrum protection: powerful against UV-A while maintaining high UV-B protection.
Provides UV-C protection (depletion of the ozone layer potentially allows more UVC rays to reach the earth's surface).
Natural Oceanic Clay works synergistically with sunscreen actives such as Zinc Oxide to achieve high SPF (UVB) and PFA (UVA) readings that cannot be achieved otherwise.
The light green tint is perfect for neutralizing the redness from pimple acne or skin irritation.
Photo-stable for long-lasting UV protection.
The light green tint can provide “skin-brightening” effect without the pasty whitening effect.
Oceanic Clay absorbs oil on the skin: ideal for those with oily skin, who usually avoid using sunscreen due to its typical heavy, oily texture. Double-blind studies shown that by only adding oceanic clay to high oil content creams significant reduce the oily skin feel

In experimentation, it has been found that the natural oceanic clay acts synergistically with other mineral sunscreen actives to achieve a high SPF & PFA that cannot be achieved otherwise. The following table is exemplary of synergism.

TABLE 1

| | | | | Increase in | | | |
|---|---|---|---|---|---|---|---|
| | | | | SPF by | | | Increase in |
| | | Natural | | adding | | | PFA by |
| | Zinc | Oceanic | SPF (in | Oceanic | | PFA (in | adding |
| Formulation Number | Oxide % | Clay % | vitro) | Clay | In vivo | vitro) | Oceanic Clay |
| A1 (without Clay) | 10% | — | 8.08 | ↑183% | | 6.79 | ↑56% |
| A2 (with Clay) | 10% | 5% | 22.86 | | | 10.62 | |
| B1 (without Clay) | 16% | — | 36.24 | ↑54% | | 10.94 | ↑125% |
| B2(with Clay) | 13% | 5% | 55.95 | | 47.30 | 24.60 | |
| C1 (without Clay) | 10% | — | 11.0 | ↑232% | | 5.35 | ↑233% |
| C2 (with Clay) | 10% | 15% | 36.5 | | | 17.85 | |
| D1 (without Clay) | 13% | — | 33.8 | ↑69% | | 19.3 | ↑68% |
| D2 (With Clay) | 13% | 2% | 57.0 | | | 32.5 | |
| E1 (Without Clay) | 8.5% | — | 14.5 | ↑106% | 16.14 | 9.9 | ↑72% |
| E2 (With Clay) | 8.5% | 2% | 29.9 | | | 17.0 | |

TABLE 2

| | | | | | Increase | | | Increase |
|---|---|---|---|---|---|---|---|---|
| | | | | SPF | in SPF by | | PFA | in PFA by |
| Sample | | Zinc | | (in | adding | In | (in | adding |
| ID | Description | Oxide % | Clay % | vitro) | Clay | vivo | vitro) | Clay |
| BN 2093 | Control | 13% | No Clay | 33.8 | — | | 19.3 | — |
| | | | (5% | | | | | |
| | | | purified | | | | | |
| | | | Water) | | | | | |
| BN 2088 | Illite Clay | 13% | 5% | 23.7 | No | | 13.5 | No |
| | | | | | increase | | | increase |
| BN 2089 | Kaolin Clay | 13% | 5% | 25.2 | No | | 14.4 | No |
| | | | | | increase | | | increase |
| BN 2090 | Bentonite | 13% | 5% | 28.1 | No | | 16.0 | No |
| | Clay | | | | increase | | | increase |
| BN 2091 | French Green | 13% | 5% | 29.5 | No | | 16.8 | No |
| | Clay | | | | increase | | | increase |
| BN 2034 | Oceanic Clay | 13% | 5% | 55.95 | ↑66% | 47.30 | 24.60 | ↑27% |
| | (80 Mesh) | | | | | | | |
| BN | Oceanic Clay | 13% | 5% | 47.3 | ↑40% | | 27.0 | ↑40% |
| 2087-C | (200 Mesh) | | | | | | | |
| BN | Oceanic Clay | 13% | 5% | 48.1 | ↑42% | | 27.4 | ↑42% |
| 2087-M | (200 Mesh) | | | | | | | |
| BN 2092 | Oceanic Clay | 13% | 2% (with | 57.0 | ↑69% | | 32.50 | ↑68% |
| | (200 Mesh) | | 3% | | | | | |
| | | | purified | | | | | |
| | | | Water) | | | | | |

All samples had a pH range between 7.5-8.5. The selection of clays included combinations of non-expandable and expandable clays with different levels of Iron Oxide, most commonly used by the cosmetic industry. These included:

### Illite Clay:

### Kaolin Clay:

### Bentonite Clay:

### French Green Clay (Montmorillonite)

### Natural Oceanic Clay:

All samples were very stable. The tested clays are known for their ability to enhance the stability of emulsions. This rules out the lower SPF due to separation of the emulsion. The test results show that no other clay has shown any SPF boosting effect. The other clays might have caused agglomeration of particles, leading to uneven distribution of Zinc Oxide and result in lower SPF & PFA.

The data set out above shows a consistent significant SPF and PFA boosting effect, tested at 2%-15% usage levels of Oceanic Clay. There is consistent SPF testing results for the same formulation from both in vitro (3 samples) and in vivo testing. Natural Oceanic Clay works synergistically with Zinc Oxide to achieve high SPF (UVB) and PFA (UVA) readings that cannot be achieved otherwise. One explanation of the Oceanic Clay's SPF boosting effect is: when used in combination with Zinc Oxide, Natural Oceanic Clay improves the dispersion of the particles, resulting in improved distribution of sunscreen actives on the skin and significantly enhanced SPF (UVB) and PFA (UVA). No other commonly used cosmetic clays tested showed any SPF boosting effect.

References may now be made to exemplary embodiments of the invention.

The following are examples of a sunscreen formulation comprising natural oceanic clay and non-nanoparticle zinc oxide. A person skilled in the art will know that a variety of other ingredients may be added to the sunscreen formulation to enhance skin care such as a moisturizer, a fragrance, a skin tint or an acne compound.

### Example #1

### Preparation of

### Natural Organic Clay Sunscreen SPF 45+

| | % Wt |
|---|---|
| | 90%-110% |
| | of value |
| Ingredient | shown |
| Cocoglucoside and Coconut Alcohol | 3.50% |
| Arachidyl Alcohol and Behenyl Alcohol and | 2.50% |
| Arachidyl Glucoside | |
| Simmondsia Chinensis (Jojoba) Seed Oil | 1.00% |
| Caprylic/Capric Triglyceride | 13.00% |
| Dimethicone | 5.00% |
| Zinc Oxide | 13.00% |
| Purified Water | 50.97% |
| Sodium Magnesium Silicate | 0.30% |
| Xanthan Gum | 0.20% |
| Methylcellulose | 2.00% |
| euterpe oleracea (acai) fruit extract | 1.00% |
| Sea Silt (Natural Oceanic Clay) | 3.00% |
| Water (and) Algae Extract | 3.00% |
| Cupressus sempervirens (Cypress) Oil | 0.03% |
| Citrus Aurantium Bergamia (Bergamot) Fruit Oil | 0.50% |
| Benzyl Alcohol, Salicylic Acid, Glycerin & Sorbic Acid | 1.00% |

### Example #2

### Preparation of

### Natural Organic Clay Sunscreen SPF 45+

| | % Wt |
|---|---|
| | 90%-110% |
| | of value |
| Ingredient | shown |
| Cocoglucoside and Coconut Alcohol | 3.50% |
| Arachidyl Alcohol and Behenyl Alcohol and Arachidyl | 2.50% |
| Glucoside | |
| Simmondsia Chinensis (Jojoba) Seed Oil | 1.00% |
| Caprylic/Capric Triglyceride | 13.00% |
| Dimethicone | 5.00% |
| Zinc Oxide | 13.00% |
| Purified Water | 48.97% |
| Sodium Magnesium Silicate | 0.30% |
| Xanthan Gum | 0.20% |
| Methylcellulose | 2.00% |
| euterpe oleracea (acai) fruit extract | 1.00% |
| Sea Silt (Natural Oceanic Clay) | 5.00% |
| Water (and) Algae Extract | 3.00% |
| Cupressus sempervirens (Cypress) Oil | 0.03% |
| Citrus Aurantium Bergamia (Bergamot) Fruit Oil | 0.50% |
| Benzyl Alcohol, Salicylic Acid, Glycerin & Sorbic Acid | 1.00% |

In other embodiments of the invention the following inorganic sunscreen actives can be used in the quantities shown in the following Table 3.

TABLE #3
Inorganic Sunscreen Actives

| Proper name(s) | Common name(s) | Source material(s) | Quantity |
|---|---|---|---|
| Titanium dioxide | Titanium dioxide | Titanium dioxide* | ≦25% |
| Zinc oxide | Zinc oxide | Zinc oxide* | ≦25% |

In still further embodiments of the invention the following organic sunscreen actives can be used in the quantities shown in the following Table 4.

TABLE #4
Organic Sunscreen Actives

| Medicinal ingredient | Synonyms and | |
|---|---|---|
| preferred name | other recognized names | Quantity |
| Avobenzone¹ | Butyl methoxy dibenzoylmethane | ≦5% |
| Cinoxate | 2-Ethoxyethyl p-methoxycinnamate | ≦3% |
| Diethanolamine- | | ≦10% |
| methoxycinnamate | | |
| Dioxybenzone¹ | Benzophenone-8 | ≦3% |
| Drometrizole | | ≦15% |
| trisiloxane¹ | | |
| Ensulizole | 2-Phenylbenzimidazole-5-sulfonic acid | ≦8% |
| Enzacamene | 4-Methylbenzylidene camphor | ≦6% |
| Homosalate | Homomenthyl salicylate | ≦15% |
| Meradimate¹ | Menthyl 2-aminobenzoate,menthyl | ≦5% |
| | anthranilate | |
| Octinoxate | 2-Ethylhexyl methoxycinnamate, octyl | ≦8.5% |
| | methoxycinnamate | |
| Octisalate | Octyl salicylate2-ethylhexyl salicylate | ≦6% |
| Octocrylene | 2-Ethylhexyl-2-cyano-3,3 | ≦12% |
| | diphenylacrylate | |
| Oxybenzone¹ | Benzophenone-3 | ≦6% |
| Padimate-O | Octyl dimethyl PABA | ≦8% |
| Sulisobenzone¹ | Benzophenone-4 | ≦6% |
| Terephthalylidene | 3,3′-(1,4-phenylenedimethylidene) | ≦10% |
| dicamphor sulfonic | bis[7,7-dimethyl-2-oxobicylclo[2.2.1] | |
| acid¹ | hept-1-yl methanesulfonic acid | |
| Triethanolamine | Trolamine salicylate | ≦12% |
| salicylate | | |

What is claimed is:

1. A sunscreen formulation including synergistically effective amounts of natural oceanic clay and at least one inorganic sunscreen active.

2. The sunscreen formulation of claim 1 including at least one organic sunscreen active.

3. The formulation of claim 1 wherein said at least one inorganic sunscreen active is zinc oxide.

4. The formulation of claim 1 wherein the at least one inorganic sunscreen active is titanium dioxide.

5. The formulation of claim 1 wherein said natural oceanic clay is a marine glacial clay.

6. The formulation of claim 5 wherein said marine glacial clay is a chlorite-mica clay.

7. The formulation of claim 6 wherein said chlorite-mica clay comprises naturally occurring silica, iron oxide, titanium dioxide and zinc oxide.

8. The formulation of claim 1 wherein the natural oceanic clay is present in an amount of about 3% by wt.

9. The formulation of claim 1 wherein the natural oceanic clay is present in an amount of about 5% by wt.

10. The formulation of claim 1 wherein the natural oceanic clay is present in an amount of between 2% and 15% by wt.

11. The formulation of claim 3 wherein the zinc oxide is present in an amount of about 13% by wt.

12. The formulation of claim 3 wherein the zinc oxide is present in an amount of less than 25% by wt.

13. The formulation of claim 3 wherein the zinc oxide is non-nanoparticle.

14. The formulation of claim 4 wherein the titanium dioxide is present in an amount of less than 25%.

15. The formulation of claim 1 wherein the natural oceanic clay is 200 mesh.

16. The formulation of claim 1 wherein the natural oceanic clay is between 80 mesh and 200 mesh.

17. A sunscreen formulation having a sun protection factor greater than 45 including oceanic clay in an amount of between 2% and 15% by wt and non-nanoparticle zinc oxide in an amount of 13% by wt.

18. The sunscreen formulation of claim 17 further including a pharmaceutical active comprising an anti-acne, anti-dermatitis, anti-fungal and anti-inflammatory.

19. The sunscreen formulation of claim 17 further comprising an organic sunscreen active in a synergistically amount.

20. A water based sunscreen formulation having a sun protection factor greater than 45 including, in synergistically active amounts, oceanic clay, a non-nanoparticle inorganic sunscreen active, a fragrance, a pharmaceutical active topical, a moisturizer and a tint.
